(19) United States
(12) Patent Application Publication
(10) Pub. No.: US 2021/0322334 A1
(43) Pub. Date: Oct. 21, 2021

(54) CHITOSAN GELS (A) CONTAINING METAL NANOPARTICLES OF COPPER, SILVER AND ANTIBIOTICS (CIPROFLOXACIN, CEFOTAXIME, GENTAMICIN AND CLOXACILLIN)

(71) Applicant: CARDENAS Y CIA LTDA, Coronel Concepción (CL)

(72) Inventor: Galo Cárdenas Triviño, Coronel Concepción (CL)

(73) Assignee: CARDENAS Y CIA LTDA, Coronel Concepción (CL)

(21) Appl. No.: 16/973,000

(22) PCT Filed: Jun. 7, 2018
(86) PCT No.: PCT/CL2018/050042

§ 371 Date: Jul. 5, 2021

(51) Int. Cl. A61K 9/51; A61K 31/702; A61K 31/496; A61K 31/431; A61K 31/546; A61K 47/02; A61P 31/04; A61K 9/50; A61K 9/06; A61K 47/36

(52) U.S. Cl. CPC A61K 9/5161; A61K 31/702; A61K 31/496; A61K 31/431; A61K 47/36; A61K 47/02; A61P 31/04; A61K 9/501; A61K 9/06; A61K 31/546

(57) ABSTRACT

The present invention describes chitosan gel (poly-beta-glucosamine) having bacteriocidal and fungicidal properties in a mixture with nanoparticles of copper and/or silver, and/or antibiotics (ciprofloxacin, cloxacillin, gentamicin and cefotamine, and a mixture of ciprofloxacin and cloxacillin, gentamicin and cefotaxim), and a process of obtaining and using the described gel.

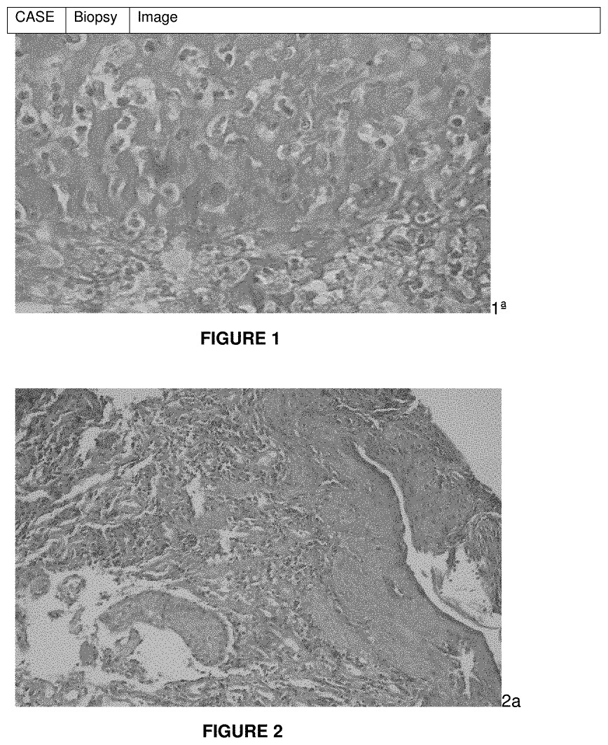

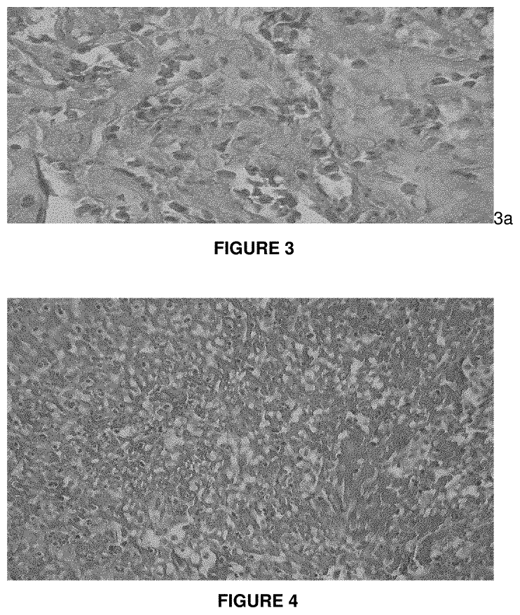

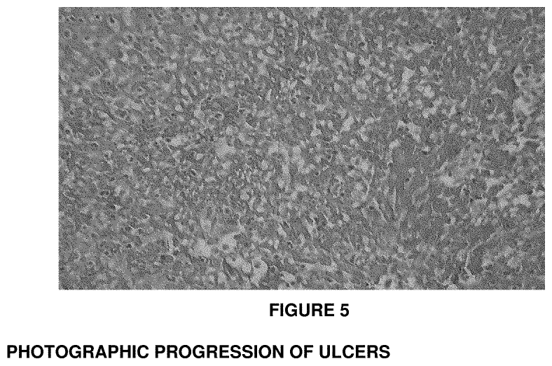

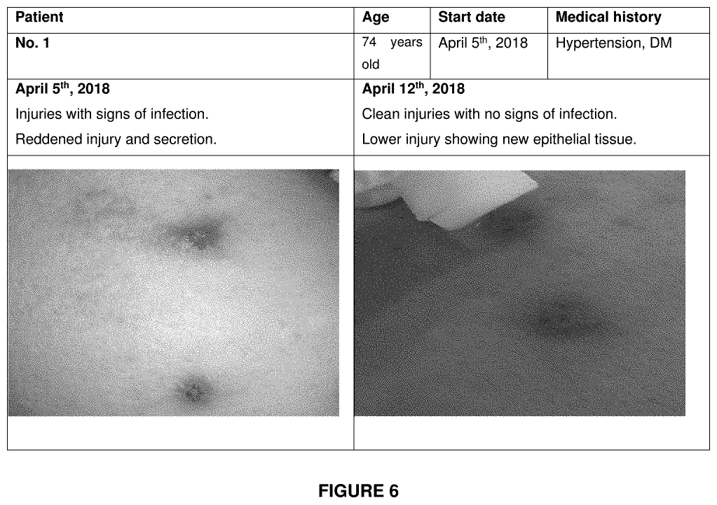

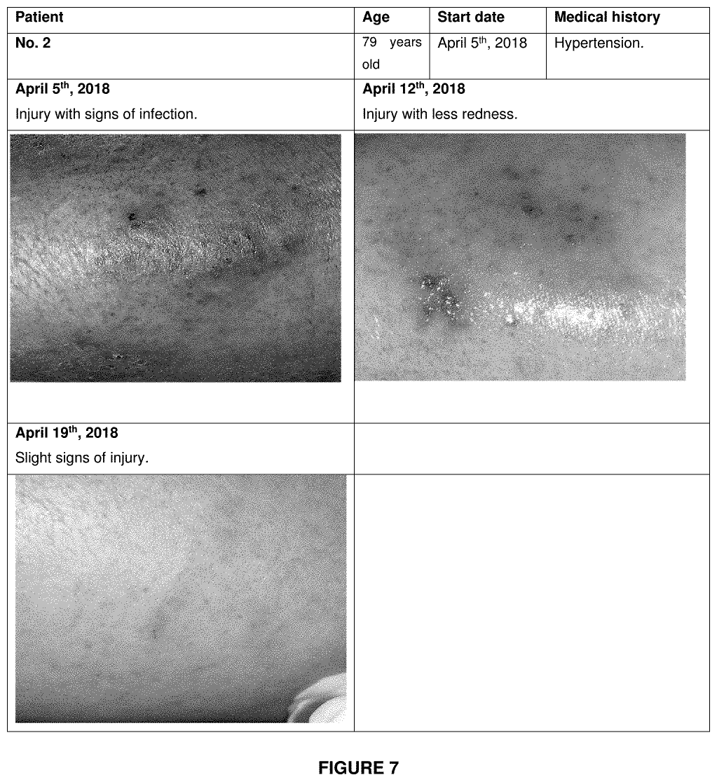

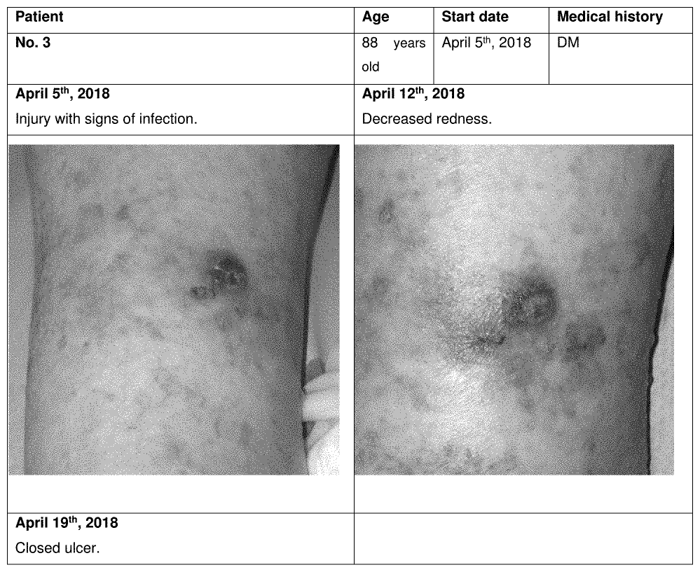

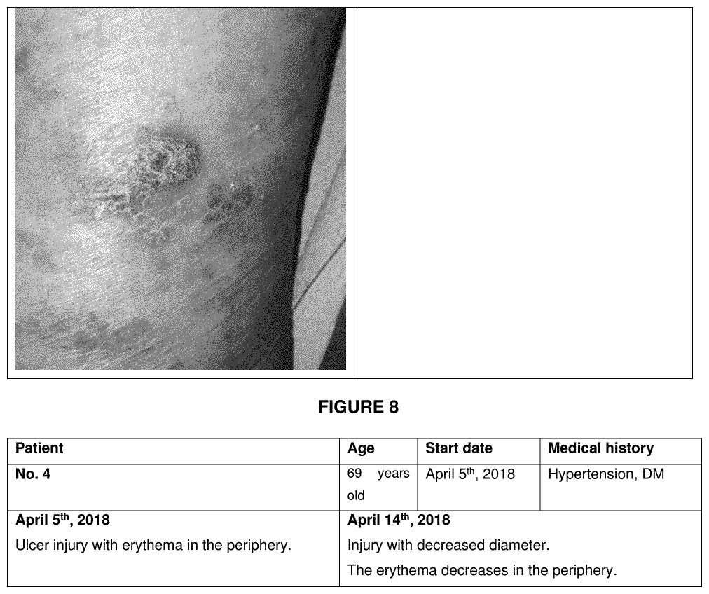

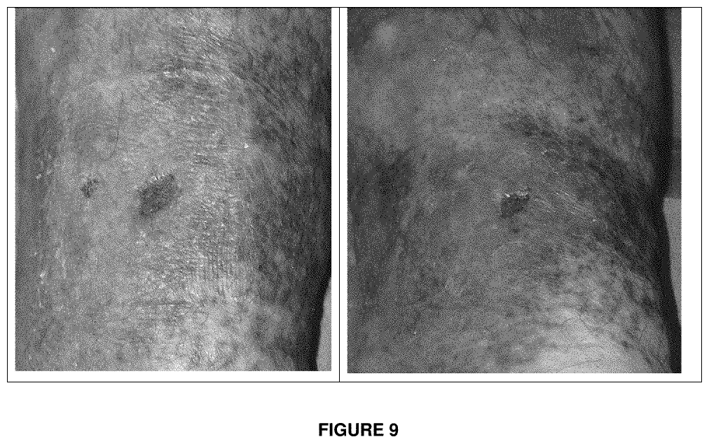

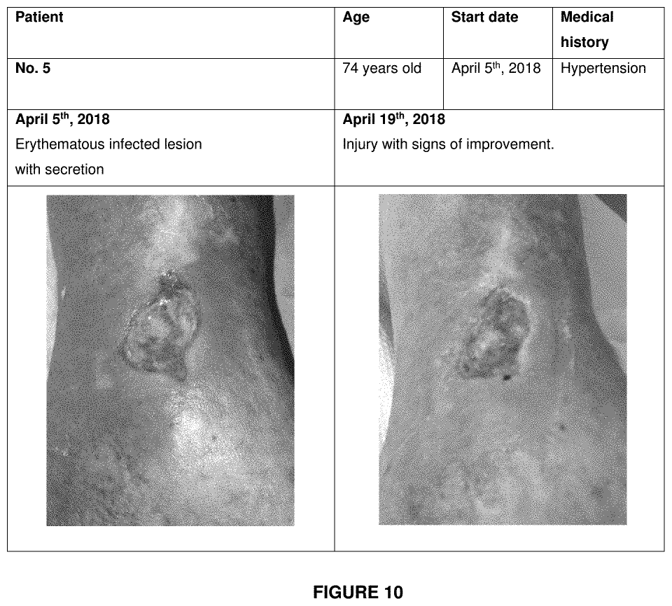

### TECHNICAL FIELD

The technical field corresponds to medicine and biotechnology, more specifically to the treatment of skin infections, especially bacterial and fungal ones such as neuropathic ulcers or diabetic foot; said treatment is also useful in wounds that require restoration of epidermal and dermal tissues, neovascularization and histopathological neoangiogenesis.

### BACKGROUND OF THE INVENTION

There is a problem in health care centers for the treatment of skin infections (neuropathic ulcers, diabetic foot and others) caused by bacteria.

The skin covers the body surface forming a protective barrier against the action of chemical, microbial or physical agents on deeper tissues.

The skin is an essential tissue controlling the environment stability, it is formed by two layers of tissues—an epidermis, which is a superficial layer, and a dermis, that is a deep layer. The epidermis is a multilayered epithelial tissue of cells originated in the so-called basal or germinative stratum, which is also known as the Malpighi layer, formed by living cells that reproduce continuously, displacing the younger cells to the older ones, which die and are loaded with a protein called keratin, and end up being detached. The inner layer is the dermis, a highly vascularized connective tissue that contains several types of sensory receptors, such as those for the sense of touch, temperature and pain. The mucous membranes of the different channels come next. It consists of a network of collagen and elastic fibers, blood capillaries, nerve, fat lobes and the base of the hair follicles and sweat glands.

The skin plays an important role in maintaining body temperature thanks to the action of the sweat glands and blood capillaries. That is to say, it is fundamental in the homeostasis of the organism.

This important organ, which is one of the largest in the body (1.5-2.4 m²) is exposed to many wounds that result in continuity problems or marked loss of substance.

Additionally, the open surface allows the microbial invasion. As a result, most of the deaths occur during the first week as a product of the shock (1, 2).

The patient is hospitalized for the management of the acute and sub-acute stage of the skin lesion. Depending on the seriousness of the patient's condition or if she/he also presents some underlying pathology (epilepsy, alcoholism, psychiatric disorders, diabetes, etc.) she/he is hospitalized for treatment with high costs in the health system and a very slow recovery.

Diabetic Foot

Infections associated with the diabetic foot are subdivided into two groups—superficial infections and deep ones. Superficial infections present as localized cellulitis, but the infections range can include necrotizing fasciitis in deep infections. The microorganisms involved in these infections include Staphylococcus aureus, Streptococcus spp., Pseudomonas aeruginosa, Acinetobacter baumannii, enterobacteria and strict anaerobic bacteria. Isolation of Stenotrophomona maltophila and methicillin-resistant S. aureus has also been reported as an etiological agent in hospitalized patients.

Diabetes mellitus in most of the western world and in intermediate developing countries has acquired epidemic characteristics, resulting in a series of chronic complications that include—among others, coronary heart disease, diabetic retinopathy, diabetic neuropathy, diabetic nephropathy, atherosclerosis and neurovascular compromise of the limbs that leads to the occurrence of lesions in the foot with formation of ulcers that are hard to manage.

As a result of anatomical deformity, evident alteration of macro- and microcirculation, neuropathy—among others, the increase of skin damage and soft tissue is facilitated without the patient necessarily being aware of this situation. This is known as diabetic foot.

All of these complications are associated with significant morbidity and mortality that make the care of these patients more expensive worldwide.

The diabetic foot corresponds to a late complication in the natural history of diabetes mellitus, which occurs due to the destruction of the skin barrier, infectious compromise of skin, soft parts, fascias, tendons and finally bone. From a microbiological standpoint these infections when superficial are typically produced by strains of Staphylococcus spp. and Streptococcus spp.; however, as they progress and deepen they are typically polymicrobial and mixed ones.

The usual management of these patients requires a multidisciplinary approach mainly given by vascular surgery, which will try on the basis of the angiographic study the strategy of performing revascularization surgeries in the affected area, trying to limit as much as possible the performance of amputation surgeries; the endocrinologist, who will try to compensate the patient from the metabolic point of view and the infectious disease specialist, who will adapt the antimicrobial therapy according to the own patient's characteristics, depth of the lesion, microbiological isolates and epidemiological context.

In general a very important number of patients will require surgeries with limb amputation at different levels, resulting in important limitations in the patient's quality of life; on the other hand, the selection of antimicrobial therapy is strongly limited by poor tissue vascularization and the growing description of antibiotic resistant bacteria.

In this sense, it is explained that in the diabetic foot different therapeutic alternatives are being tried, which are distinct from the traditional ones in their handlings that include the use of biologically-treated larval forms of insects that remove the necrotic tissue of this type of lesions, hyperbaric chamber to increase the supply of oxygen and to facilitate the destruction of anaerobic bacteria, among others.

Accordingly, any strategy that tries to facilitate bacterial eradication on the one hand and/or to facilitate the regeneration of strongly devitalized tissue on the other hand requires a thorough investigation to establish a possible coadjutant role in the treatment of this type of pathology.

Foot problems are present in more than 15% of diabetics. The diabetic foot is the complication that generates the greatest number of hospitalizations in the diabetic population, being also recognized as the main cause of prolonged hospitalization in medical and general surgery rooms. It occurs in both type 1 and type 2 diabetes, with a prevalence of 5.3% to 10.5%. The diabetic presents an accumulated risk of ulceration of 15% during her/his life (3). The diabetic foot corresponds to the first cause of major amputations of non-traumatic origin, wherein diabetics present a risk 10 times greater than the general population to require an amputation—with rates reaching 2.8% of all diabetics.

The international frequency recognized in developed countries regarding diabetic foot ulceration corresponds to an accumulated of 5.8% to 3 years, requiring an amputation in 15% thereof.

About 15% of all patients suffering from diabetes mellitus will develop a foot or leg ulcer during the course of their disease. The magnitude of said numbers is evidenced by the fact that more than 25% of the hospital admissions of diabetics in the USA and Great Britain are related to problems in their feet. In economic terms, this problem causes in the USA an annual cost of about one trillion dollars.

Venous Ulcers Venous ulcers are one of the most prevalent diseases in older adults, mainly hypertensive and diabetic patients, and said ulcers are complex wounds that instead of healing become permanent injuries in the lower extremities, and only with the use of latest generation dressings, elastic compression systems and drug treatment they can be healed in a short time improving the quality of those who suffer from them.

Venous hypertension ulcers constitute the largest percentage (between 75% and 90%) of all vascular ulcers. Due to their high incidence (between 2% and 3% of the population) the care thereof constitutes 50% of total nursing time in primary care, according to some studies.

Chronicity and relapse are their most relevant clinical characteristics, half of these ulcers remain open over nine months, 20% are open until two years, and 10% until five years, reappearing a third of the initially healed ulcers within twelve months of healing.

Arterial Ulcers

Arterial ulcers can be defined as those that are a consequence of a deficit of blood supply in the affected limb, which is secondary to a generally chronic arteriopathy. They are also known as “ischemic” ones.

They tend to have a chronic progression, with a poor prognosis due to the poor therapeutic response and the concomitant systemic processes in the patients, in addition to a high risk of infection.

The treatment is complex as the origin of the problem is an arterial occlusion and as long as the circulation is not restored it will be difficult to cure; therefore, the best attitude is prevention.

According to various studies, arterial ulcers would correspond to 10%-25% of all vascular ulcers, and they mostly affect men over 50 years of age with peripheral obliterating arteriopathy. Diabetes and especially smoking are considered high risk factors for the onset thereof.

Their preferential location is in distal areas or on the anterior-lateral side of the leg, over bone prominences, points subjected to pressure in the feet, fingertips, interdigital areas, heel, metatarsal heads, among others.

As other causes of arterial ulcer, diabetic macro and microangiopathy, thromboangiitis obliterans (Buerger's disease), hypertensive ischemia or acute arterial embolisms are observed.

Due to the fact that there is no product that allows the recovery of this type of wounds, it is pertinent to elaborate a gel that controls the infection and allows a recovery of wounds caused by this disease.

Chitosan Activity on Cellular Tissue

It has been proved that chitosan can be integrated at the level of animal and microbial cells. This property of chitosan can lead to a variety of biomedical applications such as coagulation enhancers, bacteriostatic and sperm-killing agents (4).

Chitosan as Hemostatic Agent

Chitosan is a polycation. Since the 1950's polycations have been known to bind to blood cells and are effective binding agents. In the 1960s, chitosan was investigated due to its binding abilities. It was found that Chitosan—even at very low concentrations, has the ability to agglutinate platelets. Accordingly, it can be considered as a pro-coagulant agent (4). The agglutination of platelets by polycations is dependent on both the structure of the polymer and the molecular weight thereof.

Besides the six most common polycations, only chitosan is able to effectively initiate the formation of a heparinized blood gel. Chitosan having a molecular weight of 35,000 g/mol is capable of producing a weak clot in heparinized blood, while chitosan having a molecular weight of 600,000 g/mol (4) produces a very firm clot.

Synthetic skin: at the University of Illinois (USA) synthetic skin has been prepared to cover substance losses such as burns and ulcers. This coating is a mixture of three biopolymers, chitosan acetate, quercetin ammonium salt (from sheep's wool) and collagen acetate (from bovine hides). We prepare Biopiel™ using chitosan and fatty acids.

zo Surgical sutures: chitin-chitosan in the form of fibers can serve as absorbable sutures of animal origin (5).

Ophthalmological applications: contact lenses have been prepared from chitosan, which are more biocompatible than the ones obtained from synthetic polymers. They are more malleable, air permeable and water absorbent (6).

Artificial biomembranes to encapsulate enzymes: chitosan—a cationic polymer, and alginate—an anionic polymer obtained from algae, interact to form a water-insoluble membrane used to encapsulate insulin (7, 8).

In the field of membranes:

Wang and Spencer (9) report studies of chitosan-based ultrafiltration membranes using titanium dioxide as a porous substrate.

Tomasewska (10) reports on chitosan membranes having potential use in kidney dialysis.

Another industrial and biological application is the manufacture of hydrogels. Hirano and Usutani (11) report on hydrogels prepared from aqueous solutions that are useful in the area of medicine, specifically in the treatment of wounds.

An important property of the polymers under development is their solubility (12). For chitin the most used systems are the following ones. (a) N,N-dimethylacetamide (DMAc) -5% LiCI and (b) N-methyl-2-pyrrolidone (NMP)-5% LiCI.

Regarding other biological properties there are some examples such as the following ones.

Tokura et al. (13) developed biological materials based on chitosan and derivatives having antibacterial and biodegradable properties.

Postieszny (14) disclosed that chitosan controls the transmission of PSTV.

Staroniewicz et al. (15) studied:

a) bactericidal effect on: Escherichia coli, Staphylococcus aureus, Pseudomonas aeruginosa, and Salmonella Paratyphi B.

b) fungicide effects on: Candida albicans, Trichophyton mentagrophytes, and Micorosporum canis.

In all cases the species were sensitive to chitosan. Andrews determined the minimum inhibitory concentrations of various bacteria (16).

Chitosan Regarding Healing

It has been described that chitin (poly-N-acetyl-glucosamine) accelerates the healing process by decreasing the recovery period in some cases of burn injuries.

Chitin has been applied on natural-origin sutures such as silk and catgut (intestinal submucosa) and the incorporation thereof shows a behavior with less tissue inflammatory reaction; the same was observed when adding chitin on healing dressings (17).

Chitosan in Wound Care

On the other hand, chitosan (poly-Beta-D-glucosamine) is a very attractive candidate for the care of bloody wounds, especially the ones generated by burns. The mechanism is explained as follows: chitosan can form biocompatible and absorbent films. The application thereof can be carried out as a pre-made film or a liquid application directly on the lesion, which will form the film. This film is permeable to oxygen, positively antagonizing the hypoxia of the affected tissues (18).

As the pH thereof is slightly acidic, it provides a refreshing and anesthetic sensation.

Finally, since it is degraded by organic enzymes, it does not need to be removed manually, thus avoiding painful maneuvers for the patient.

Nanoparticles

Nanotechnology is a new branch of science that specializes in studying and manipulating matter at atomic level. This term was first coined in a conference given in 1959 by the American physicist Richard Feynman—considered the father of nanoscience, who stated that problems in the area of physics, chemistry and biology could be solved if we were able to see what we are doing at the atomic level, thus proposing to manufacture products based on a rearrangement of atoms and molecules. Thus, for the first time, a new scientific discipline is introduced, which is promising in the modern era: “Nanotechnology”. Since then, scientists from all over the world have observed the behavior of matter on a nanometric scale, thus opening a perspective to new knowledge that was never explored before (19). In 1980, Eric Drexler suggested the possibility of creating molecular-scale engineering systems, as he stated, “Everything is related to the way atoms are ordered: coal and diamonds, sand, glass, cancer and healthy tissue”; are examples of pairs of materials made up of the same atoms; however, variations in the order thereof make the difference (20).

The nanoparticles (NPs) are of the order of the nanometer or thousandth of a micron, and the number of atoms that compose said NPs are of the order of hundreds. Nowadays, different experimental techniques are used, which allow the study, design, creation, synthesis, manipulation, characterization and application of NPs. The new physical properties detected on different types of NPs have awakened scientific curiosity for possible applications in medicine, thus originating a new concept named “Nanomedicine”, which corresponds to one of the most promising branches within the potential new technological advances in this field. One could venture a definition as “a branch of nanotechnology that would allow the possibility of curing diseases from inside the organism at a cellular and/or molecular level”.

It is considered that certain fields can be subjected to a real revolution, especially the following ones: monitoring, tissue repair, control of diseases progression, defense and improvement of human biological systems, diagnosis, treatment and prevention of diseases, pain relief, selective administration of drugs to affected cells, among others. All this would constitute new technological advances in health, which would position it in a new scientific and healthcare era. The use of these NPs having magnetic properties can implicate a significant impact on phenomena such as hyperthermia and selective drug transport, both of which have a promising future in cancer therapy, as well as applications based on cellular endocytosis, through which the cell captures and incorporates magnetic NPs (NPMs) (21).

In the last decade, an exponential growth has been observed concerning the development and approval by the regulatory authorities of drugs, hybrid therapeutic nanosystems and drug transport with clinical application mostly as oncological therapy. These therapeutic nanosystems include, for example, liposomes, monoclonal antibodies and immunoconjugates, NPs or therapeutic polymers such as polymer drugs or polymer-protein conjugates. These often multi-component nanoconstructions can be defined as the first nanomedicines having demonstrated clinical benefit, although—in reality, this concept is not completely new, since immunoconjugates, liposomes, NPs or polymer conjugates were already known in the 1970s; however, they were considered as individual and competitive technologies. Nanodevices are 100 to 100,000 times smaller than human cells. As a reference, the head of a pin is one million nanometers wide, a human hair about 80,000 nanometers in diameter, while a DNA molecule is 2 to 12 nanometers wide.

In recent years, silver has generated much interest due to its good conductivity, chemical stability and its catalytic and antibacterial activity. AgNPs are becoming one of the fastest growing product categories in the nanotechnology industry according to a market research carried out by Bourne (22). The strong antimicrobial activity thereof is the main feature for the development of products with AgNPs; currently, a wide category of products are available in the market. In the medical field, there are wound dressings, contraceptive devices, surgical instruments and bone prostheses, all coated or integrated with AgNPs to prevent bacterial growth (Cheng et al.) (23); Cohen et al. In addition, the use of AgNPs is also being evaluated against the treatment of diseases that require a maintained concentration of drug in the blood or with specific targeting to cells or organs, Panyman et al. (25), as with the HIV-1 virus, since in vitro treatment with AgNPs has been shown to interact with the virus and inhibit its ability to bind to host cells, Elechiguerra et al. (26). In daily life consumers may come into contact with AgNPs contained in aerosols, detergents, refrigerators, washing machines, pacifiers, water purification systems, wall paints and cosmetic products, Zhang and Sun (27).

AgNPs are also incorporated in the textile industry for the manufacture of clothing, underwear and socks, Lee et al. (28). By introducing AgNPs into synthetic or natural fibers, they enhance the ionic activity due to the higher amount of silver ions that are released. As a result, rapid antimicrobial or anti-odor effects are achieved.

Gels

In current therapeutics, semi-solid formulations of antibiotics are preferably used in the treatment of wounds, whether these are caused by thermal damage, surgical one or other damages. The effect of these drugs reside in their antimicrobial activity, preventing or controlling septic complications, which are the main cause of delaying the normal healing process. Among these topical agents are silver sulfadiazine cream, bacitracin ointment, 0.5% AgNO3 solutions, nitrofurazone, etc. In spite of the therapeutic incidence of this group of drugs in the cure of these affections, none of them contributes directly in the tissue reconstruction.

Application AU2016335462 describes the use of silver-ion charged nanoparticles to treat microbial infections, without contributing to tissue regeneration and without considering the use of antibiotics.

Accordingly, of all the possible applications of chitosan, biomedical is the most promising one, due to the great beneficial effects it would have on human health, and at a very low cost, specifically highlighting the regenerative activity of damaged tissue. This added to nanotechnology and antimicrobials, such as metal ions and/or antibiotics, would solve a technical problem in the area of medicine that affects millions of people worldwide.

### DETAILED DESCRIPTION OF THE INVENTION

The present application describes the preparation of topical use gels containing a chitosan matrix and nanoparticles charged with metallic nanoparticles, metallic ions and/or antibiotics; these components provide the gels with tissue regenerating and antimicrobial properties, which are useful for use on skin suffering from lesions and/or bacterial infections.

Method of Production

Step 1—Elaboration and Characterization of Gel Formulations Synthesis of Gels

i) Preparation of chitosan gels

The formulations will be prepared according to classic techniques of pharmaceutical technology for the preparation of semi-solid pharmaceutical forms.

A chitosan polymer with a deacetylation degree higher than 90% and a molar mass between 25,000 and 500,000 g/mol is used, which is obtained by dissolution at room temperature (15-25° C.) in weak organic acids (acetic acid, formic acid, lactic acid, among others) until a concentration between 1 and 5% by mass is achieved.

ii) Preparation of chitosan mixed with antibiotics

One or more antibiotics are added to a chitosan matrix in a concentration of 0.1 to 5% by mass and in a ratio of 1:1 if there is a combination of antibiotics, by means of the method of dispersion and agitation in gaseous nitrogen atmosphere.

iii) Preparation of chitosan gel doped with metallic nanoparticles
a) Co-deposition reaction: this method involves the physical co-deposition of metal vapors with organic vapors (2-ethoxyethanol) in liquid nitrogen, in a reactor of metal atoms; the metal to be evaporated is introduced into an alumina crucible; the magnet and the solid chitosan are introduced into the bottom of the reactor; the flask is connected to the solvent to be used (2-ethoxyethanol) and the whole system is evacuated until a high vacuum of 10⁻⁵ bar is reached. The reactor is then immersed in a 5000 ml Dewar with liquid nitrogen. The power source is turned on up to 40A, which evaporates both the solvent and then the metal at its respective boiling temperature, penetrating the solvent into the reactor in vaporous state. The power source is turned off, the metal and the solvent are co-deposited in a process called nucleation for a period of approximately 60 minutes, finally forming a frozen “metal-organic” matrix on the internal walls of the reactor (Chemical Liquid Deposition).
b) Preparation of chitosan doped with nanoparticles: the chitosan doped with the metallic nanoparticles is obtained by defrosting the “metal-organic” matrix found in the internal walls of the reactor. The matrix is shaken for 12 hours with a magnetic stirrer, and it rests for a period of approximately 2 hours, thus obtaining the “metal-organic-polymer” matrix from which it is extracted by connecting a receiving flask to the vacuum line. Once the matrix is removed, the solvent is evaporated, which acts as a support for the nanoparticle/chitosan mixture until solid chitosan particles are obtained. These particles are doped with metallic nanoparticles. (Solvated Metal Atom Dispersion, SMAD). The concentration of nanoparticles ranges between 0.1 and 5% by mass.

iv) Characterization of chitosan doped with nanoparticles: Ag and Cu nanoparticles supported in chitosan are characterized by Transmission Electron Microscopy (TEM) and Scanning Electron Microscopy (SEM) equipped with an energy dispersive X-ray analyzer coupled to the microscope, thermogravimetric analysis (TGA) and Fourier Transform Spectroscopy (FTIR).

v) Quality Control—the antimicrobial activity of metallic nanoparticles and/or antibiotics supported in chitosan is analyzed through susceptibility tests to pathogenic microorganisms Escherichia coli, Staphylococcus aureus, Salmonella typhymorium, S. epidermidis and Candida albicans through dilution in broth and disc diffusion.

The following are examples of the preparation, application and use of antimicrobial gels, which allow a better description of the present invention, but they do not limit in any way the scope thereof.

### EXAMPLES

1. Determination of the antibacterial activity of chitosan gels with antibiotics

Chitosan gels were added with antibiotics: gentamicin, ciprofloxacin, cloxacillin and cefotaxime and two mixtures thereof. For each antibiotic two concentrations were tested—a low concentration (LC=1 mg/ml) and a high concentration (HC=10 mg/ml). Furthermore, gels of copper sulfate (2.64 mg/ml), copper oxychloride (1.5 mg/ml) and copper nanoparticles were added. The antibacterial activity of the chitos a gels with antibiotic and/or copper salts is determined by measuring the inhibition halos using an agar diffusion method. For this purpose, the microorganisms to be tested were cultured on tryptictic soy agar +5% of lamb blood for 18-24 hours at 35° C. From these cultures, a suspension adjusted to 0.5 Mc Farland was prepared in sterile distilled water and plates containing 20 ml of Mueller-Hinton agar were inoculated with a swab. Once the plates were inoculated, 6 mm diameter wells were prepared in the agar (5 per plate) with the help of a glass rod (Pasteur pipette). Approximately 30 g of antibiotic gel is placed on the wells. The plates were incubated at 35° C. for 24 hours and the inhibition halo was measured.

Six bacterial strains were used to carry out this test: Pseudomonas aeruginosa (ATCC 27853), Pseudomonas sp. (multi-resistant strain of clinical origin) Escherichia coli (ATCC 25922), Enterococcus faecalis (ATCC 29212) and two strains of Staphylococcus aureus (ATCC 29213 y ATCC 25923).

TABLE 1
Inhibition haloes (mm) of chitosan gels added with antibiotics or copper salts on different bacterial species

| | | Pseudomonas sp. | E. coli | S. aureus | E. faecal | S. aureus |
|---|---|---|---|---|---|---|
| Antimicrobial in | | ATCC 27853 | ATCC | ATCC | is ATCC | ATCC |
| chitosan gel | P. aeruginosa | Clinic* | 25922 | 25923 | 29212 | 29213 |
| Gentamicin/lactic acid/LC | 35 | 35 | 35 | 28 | 31 | 30 |
| Gentamicin/lactic acid/HC | 31 | 29 | 40 | 37 | 30 | 36 |
| Cefotaxime/lactic acid/LC | 18 | 18 | 37 | 32 | 26 | 33 |
| Cefotaxime/acetic acid/LC | 15 | 10 | 30 | 27 | 27 | 28 |
| Cefotaxime/lactic acid/HC | 41 | 40 | 45 | 31 | 34 | 30 |
| Cloxacillin/lactic acid/LC | 6 | 6 | 6 | 6 | 6 | 6 |
| Cloxacillin/lactic acid/HC | 6 | 6 | n.d. | 8 | 6 | 6 |
| Ciprofloxacin/lactic acid/HC | 40 | 40 | 40 | 32 | 32 | 30 |
| Ciprofloxacin/lactic acid/LC | 40 | 40 | 44 | 30 | 34 | 30 |
| Gentamicin-cephotaxime/acetic | 32 | 37 | 37 | 26 | 30 | 25 |
| acid/LC | | | | | | |
| Gentamicin-cephotaxime/lactic | 38 | 40 | 41 | 35 | 37 | 35 |
| acid/LC | | | | | | |
| Cloxacilin-cyprophloxacin/lactic | 40 | 40 | 44 | 30 | 34 | 30 |
| acid/HC | | | | | | |
| Copper Nanoparticle/lactic | 6 | 6 | 20 | 8 | 6 | 6 |
| acid | | | | | | |
| Copper sulfate/acetic acid | 9 | 9 | 15 | 6 | 6 | 6 |
| Copper oxychloride/acetic | 14 | 13 | 20 | 11 | 14 | 15 |
| acid | | | | | | |

*multiresistant strain

In general, it is observed that ciprofloxacin is the antibiotic that shows more activity against the tested microorganisms, having inhibition halos that ranged between 30 and 44 mm and between 30 and 46 mm for the antibiotic at low and high concentrations, respectively. Furthermore, the best activity occurred against Gram-negative bacteria. These results are consistent with the corresponding information in the literature. However, it shall be noted that there are no important differences in the diameters of the halos when comparing the antibiotic at low and high concentrations for the same microorganism.

2. Tests on humans

A descriptive, longitudinal and prospective therapeutic intervention study was conducted.

Thirty patients, who met the eligibility criteria, were included, configuring the sample for the current study. All patients started the study on the same day and remained in the study until the completion thereof. Conventional anamnesis, physical examination, nursing, clinical laboratory, anatomopathological and microbiological techniques were used for blood extraction and collection of biomaterial for biopsy and culture. Patients were treated with the combination of chitosan-silver gel and ciprofloxacin-cloxacillin gel, three times a week up to two weeks, and they were monitored for 21 days after the last therapeutic application. The application procedure was characterized by photographic record, topical application of the combined therapeutic product in the proportion of 1 mm on the whole surface of the lesion and placement of sterile dressing after full absorption of the therapeutic product. Photographic records were used to evaluate exposure to diagnosis and treatment. The outcome was evaluated by categorizing the clinical improvement of patients as a function of time. The researchers evaluated their consistency with the collected photographs to identify possible interpretation and typing errors. These inconsistencies were discussed in a group and corrected according to the data established in the photographs. From the digitalization of the photographic images, the tracking over time was framed and the information obtained was used to design a model that allowed establishing the risk that a patient has of clinical improvement. For the clinical classification of skin ulcers a technique validated in Chile in previous studies was used, which develops the following parameters, where Type 1 expresses the lowest degree of pathological affectation, whereas Type 4 shows the highest one.

| | 1 | 2 | 3 | 4 |
|---|---|---|---|---|
| ASPECT | Erythematous | Reddened | Yellow | Necrotic |
| Greater extension | 0-1 cm | >1-3 cm | >3-6 cm | >6 cm |
| Depth | 0 | >1 cm | 1-3 cm | >3 cm |
| Exudate amount | Absent | Rare | Moderate | Abundant |
| Exudate quality | No exudate | Serous | Turbid | Purulent |
| Sphacellated or necrotic tissue | Absent | <25% | 25%-50% | >50% |
| Granulatory tissue | 100-75% | 50%-<75% | 25%-<50% | <25% |
| Edema | Absent | + | ++ | +++ |
| Pain | 0-1 | 2-3 | 4-6 | 7-10 |
| Surrounding skin | Healthy | Peeled | Erythematous | Macerated |

The process of application of the therapeutic product was always preceded by asepsis actions concerning the region involved in the lesion to be treated. The study was developed as expressed by the following algorithm:

| | Days | | |
|---|---|---|---|
| Activities | 0 | 21 | 42 |
| Clinical Evaluation and | X | | |
| Selection Consultation | | | |
| Distribution of patients | X | | |
| according to age, sex | | | |
| and morbid history. | | | |
| Photographic record of the | X | X | X |
| skin ulcers and of the | | | |
| alterations in the solution | | | |
| of continuity of the skin. | | | |
| Classification of skin ulcers | X | | X |
| based on the assessment of | | | |
| macroscopic morphological | | | |
| parameters | | | |
| Application set by protocol | | | |
| of therapeutic gel I and | | | |
| therapeutic gel II (described | | | |
| in annex: product data sheet) | | | |
| Obtaining the analytical | X | | X |
| parameters | | | |
| Obtaining the anatomopathological | X | X | X |
| and microbiological parameters | | | |

Description and operationalization of variables

| | | | Operational | | |
|---|---|---|---|---|---|
| Dimension | Variable | Classification | definition | Scale | Indicator |
| Sociodemographic | Age | Qualitative | In exact years | a) 18-37 years old | Absolute |
| and morbid | | Ordinal | completed according | b) 38-55 years old | frequency |
| history | | Polytomous | to date of birth at the | c) ≥56 years old | and percentage |
| | | | time of the study. | | |
| | Sex | Qualitative | It was considered | a) Male | Absolute |
| | | Nominal | according to the | b) Female | frequency |
| | | Dichotomous | condition of the | | and percentage |
| | | | individual determined | | |
| | | | by the characteristics | | |
| | | | biologically given by | | |
| | | | the genes. | | |
| | AH | Qualitative | The presence of AH in | a) Yes | Absolute |
| | | Nominal | a patient was | b) No | frequency |
| | | Dichotomous | considered, if she/he | | and percentage |
| | | | was previously | | |
| | | | diagnosed by her/his | | |
| | | | doctor, if she/he was | | |
| | | | being treated with | | |
| | | | antihypertensive | | |
| | | | drugs, or if the | | |
| | | | diagnosis was made | | |
| | | | during hospital | | |
| | | | admission according to | | |
| | | | the criteria proposed | | |
| | | | by the Joint National | | |
| | | | Committee.⁷ | | |

| | | | Operational | | |
|---|---|---|---|---|---|
| Dimension | Variable | Classification | definition | Scale | Indicator |
| Sociodemographic | DM | Qualitative | The presence of DM in | a) Yes | Absolute |
| and morbid | | Nominal | a patient was | b) No | frequency |
| history | | Dichotomous | considered, if she/he | | and percentage |
| | | | was previously | | |
| | | | diagnosed by her/his | | |
| | | | doctor, if she/he was | | |
| | | | being treated with | | |
| | | | hypoglycemic drugs, or | | |
| | | | during the investigation | | |
| | | | the diagnosis was | | |
| | | | made according to the | | |
| | | | criteria of the American | | |
| | | | Diabetes Association.⁸ | | |
| | Peripheral | Qualitative | The set of syndromic, | a) Yes | Absolute |
| | arterial | Nominal | acute or chronic | b) No | frequency |
| | disease | Dichotomous | conditions was | | and percentage |
| | | | considered, which are | | |
| | | | generally derived from | | |
| | | | the presence of an | | |
| | | | occlusive arterial | | |
| | | | disease that conditions | | |
| | | | an insufficient blood | | |
| | | | flow to the limbs ⁹ | | |
| Sociodemographic | Peripheral | Qualitative | The set of syndromic, | a) Yes | Absolute |
| and morbid | venous | Nominal | acute or chronic | b) No | frequency |
| history | disease | Dichotomous | conditions was | | and percentage |
| | | | considered, which are | | |
| | | | generally derived from | | |
| | | | the presence of an | | |
| | | | occlusive venous | | |
| | | | disease that conditions | | |
| | | | the imbalance | | |
| | | | established at the | | |
| | | | microcirculation level | | |
| | | | between some factors | | |
| | | | favoring the venous | | |
| | | | return and other factors | | |
| | | | hindering the same. | | |
| Basic | Analytical | Quantitative | Analytical data of | Numerical | Mean and |
| background | biological | Continuous | hematological, renal and | values | Standard |
| of the | parameters | | hepatic function | | Deviation |
| organism's | | | (Leukocytes, Urea, | | |
| baseline | | | Creatinine, GOT, GPT, | | |
| | | | GGT) obtained through | | |
| | | | clinical laboratory | | |
| | | | methods were | | |
| | | | considered. | | |
| Classification | Skin ulcer | Qualitative | It was considered from | a) Type 1 | Absolute |
| of skin | | Ordinal | the application of a | (score10-15) | frequency |
| ulcer | | Polytomous | validated instrument | a) Type 2 | and percentage |
| | | | that typifies ulcers | (score16-21) | |
| | | | from the result of the | a) Type 3 | |
| | | | general score | (score 22-27) | |
| | | | originated from an | c) Type 4 | |
| | | | assessment by | (score 28-40) | |
| | | | parameters between 1 | | |
| | | | and 4 points of | | |
| | | | macroscopic | | |
| | | | morphological aspects | | |
| | | | (appearance, greater | | |
| | | | extension, depth, | | |
| | | | amount of exudate, | | |
| | | | quality of exudate, | | |
| | | | sphacellated or | | |
| | | | necrotic tissue, | | |
| | | | granulatory tissue, | | |
| | | | edema, pain, | | |
| | | | surrounding skin) | | |
| | | | explored at the site of | | |
| | | | injury. | | |

Temporary profile Conceptual definition: time was defined as the days between the first and last control photograph.

Anatomopathological profile Conceptual definition: The preparation of a pathological anatomical report was considered as such, wherein said report was prepared from the microscopic study of tissue obtained from the skin ulcer and obtained by means of culture that describes the characteristics of the global tissue behavior of the ulcers in the patients.

Microbiological profile Conceptual definition: The preparation of a microbiological report from the study of the tissue culture obtained from the skin ulcer was considered as such.

Erythematous or epithelial tissue Conceptual definition: pink appearance, shiny, fragile in its beginnings, it indicates that it is in the remodeling phase or by compression or burning that affects the dermis without loss of skin integrity.

Reddened tissue Conceptual definition: presence of vascularized and fragile granulation tissue indicating that it is in the proliferative phase.

Pale tissue Conceptual definition: presence of fibrin, an insoluble protein derived from fibrinogen by the action of thrombin and it is pale yellow in color. The pallor of the tissue can also be due to hypoxia (low concentration of oxygen in the tissues) or ischemia (localized deficit of blood in the tissue produced by functional vasoconstriction or obstruction of the vessels).

Necrotic tissue Conceptual definition: presence of dead, dry, hard and black tissue, although the dead connective tissue may be gray and soft.

Extension Conceptual definition: it is expressed in the largest diameter or diameter with the greater extension The measuring tools can be a circular template, acetate grid template or a flexible ruler. All of this assisted by a photographic image taking.

Depth Conceptual definition: in cavitated ulcers there may be muscle, tendon or bone involvement, being sometimes difficult to access. For the measurement thereof a swab is used to take a culture, placing it at the deepest point and measuring up to the upper edge of the wound. When the wound has sacks or lateral folds, its measurement is made according to the pointers of the clock having as base the 12, which corresponds to the head of the person who is being evaluated.

Exudate Conceptual definition: it is an important part of the wound defense mechanism. The exudate is formed on the surface as a result of fluid loss from small blood vessels, it decreases with healing and disappears with epithelialization.

Exudate amount

Rare=1-5 cc

Moderate=5-10 cc

Abundant=>10 cc

Exudate quality
Serous: clear, transparent, yellowish or pink liquid.
Turbid: formed by the mixture of the exudate from the wound healing process, and detritus from the debridement.
Purulent: thick yellowish or greenish liquid, secreted by an inflamed tissue, and composed of serum, leukocytes, dead cells and fat (cholesterol and glucose).

Sphacellated or necrotic tissue Conceptual definition: pale, hypoxic or ischemic tissue. The presence of necrotic or sphacellated tissue on the wound surface is measured as a percentage.

Granulation tissue Conceptual definition: red, moist, and fragile connective tissue that fills the wound during the proliferative phase of healing.

Edema Conceptual definition: it is the excess fluid in the tissues underlying the wound and is measured through finger pressure.

edema+=<0.3 cm

edema++=0.3-0.5 cm

edema+++=>0.5 cm

Pain Conceptual definition: Analogical scale

| | |
|---|---|
| No pain | Worst pain |

1_{——}_{——}_{——}_{——}_{——}_{——}_{——}_{——}4

Surrounding skin Conceptual definition: the skin near the wound can be modified regarding the cutaneous integrity due to mechanical effects or effects related to the inflammatory process, which can spread into the injury or have consequences.
Healthy skin=Undamaged skin
Peeling=Exfoliation of keratinized cells of variable size; silver, white or tan colored that indicates dryness of the skin and propensity to cracks and fissures.
Erythematous=Epidermis reddened due to mechanical action, pressure, skin friction or irritative dermatitis. It can be accompanied by local heat.
Macerated skin=It shows excoriations (skin lesions due to loss of very superficial substance that only affects the epidermis) and decomposition of tissues by being in contact with a humid environment.

Information Processing Techniques

The data was stored and processed automatically in the Statistical Package for Social Sciences (Version 19.0 for Windows). Absolute and percentage frequencies were used as summary measures in the qualitative variables. In the quantitative variables, the mean and standard deviation were used as a summary measure. From the digitalization of the photographic images, the tracking over time was framed and the information obtained was used to design a model that allowed establishing the risk that a patient has of clinical improvement according to the associated probability of Poisson, a confidence level of 90% was established. The statistical significance of the variation in ulcer classification before and after treatment was determined by using the Wilcoxon signed-rank test. A confidence level of 95% was established and any value of p 0.05 was considered significant for the statistician associated with the test. The results were summarized in tables and general explanatory text notes.

Results and Discussion

Thirty patients were evaluated, 10 of them were men and 60% of the patients were 58 years and older (Table 1). The time in years of the ulcers and alterations in the solution of continuity of the skin of these patients summed up to 283 years.

The ulcers were between 1 and 24 square centimeters in area. 73.3% of the patients had an ulcer with an etiology related to venous disease, followed by 13.3% whose etiology was mixed (arterial and venous), although only 46.2% of the patients reported pain, for 56.6% of these patients the pain was intense and exceeded level 6 according to the visual analog scale, and 2 patients reported a pain level of 8 and another 2 patients called the involved pain as the highest level of pain in their lives, qualifying it is with a value of 4. Diabetes mellitus was present in 40% of patients and high blood pressure in 36.6% (Table 2).

In Table 3 and graph 1 it was reported that most patients showed Type-4 skin ulcers (70%), followed by those with Type-3 skin ulcers (16.6%). It should be noted that there was only one patient with a type-1 ulcer (Table 3). Table 4 reports that after therapeutic intervention a decrease in patients of frequency of Type-4 skin ulcer (40.0%) and Type-3 (23.3%) was obtained, whereas Type-1 skin ulcers (16.6%) increased.

By conducting the Wilcoxon signed-rank Test (Table 5) it was shown that the regression of skin ulcer severity in the series of patients studied—after the therapeutic intervention, expressed in the reduction of the frequency of Type-4 skin ulcers (70.0% vs. 40.0%; p=0.01; Z=−1.342) and in the increase of Type-1 (3.3% vs. 16.3%; p=0.00; Z=−6.042) is statistically significant.

The analysis results of the contribution of the therapeutic protocol through the test of Wilcoxon signed-rank Test demonstrated that the applied intervention produced favorable changes in the clinical progression in the series of patients studied. It was evidenced that there was a contribution to the reduction of the injured area without signs of infection and with active borders (Table 5). A value of probability of 0.00 and 0.01 was achieved, which allows rejecting the null hypothesis, favoring the criterion of the clinical characteristics of the skin ulcer independently of the severity degree thereof, and said ulcers have a better evolutionary prognosis after the application of the therapeutic strategy. The model developed to establish a patient's risk of clinical improvement according to the associated probability of Poisson resulted in 0.09. This result shows that a patient suffering from a chronic ulcer of non-active borders, with presence of pain and excessive exudate, obtains control of the exudate and pain relief (directly or indirectly) after receiving the therapeutic care with AB Chitosan Gel, with a probability of 90% (0.09) of clinical improvement of her/his ulcer or affectation in the solution of skin continuity. This finding coincides with the one obtained through the Wilcoxon signed-rank Test.

These variables are important, since the pain expresses some anomaly in the healing process such as the presence of inflammation with and without infection, and maceration of the perilesional tissue. Pain, considered the fifth vital sign, should be identified and monitored in ulcers. It interferes with the closure of ulcers and wounds due to a direct decrease in blood flow. The exudate is also important, since it hinders the activity of growth factors, thus delaying the closure. Epithelialization takes place from the edges mainly, and it is dependent on an extracellular matrix that allows its migration, so it is a result of the above conditions.

In the present research, it is highlighted that the therapeutic application of chitosan AB gel did not promote infectious processes or alteration of renal and hepatic function (Table 6).

The support of the histopathological studies was relevant to confirm the clinical progression. Each case was evaluated in 3 instants: at the beginning, intermediate moment, and end of the application protocol. It was observed:
a. At the beginning: The overall description of this phase would correspond to: “Tissue fragments showing abundant changes of exudative character, with polymorphonuclear predominance and frequent pyocytes that extensively infiltrate the samples, replacing their traditional architecture with accentuated signs of tissue lysis, foci of recent hemorrhage and signs of ancient hemorrhage. Rare vascular formations of capillary character and preserved structure are observed. Devitalized material of fibrinoleucocyte and serohematic character is observed on surface cover”.
b. Intermediate moment: The overall description of this phase would correspond to:
   “Tissue fragments showing signs of connective-vascular proliferation of reparative character, characterized by the installation of congestive capillary network, chronic inflammatory infiltration of predominantly lymphoplasmacytic complemented with less polymorphonuclear component. No devitalized material or signs of microorganisms are observed.
c. End of the protocol: The overall description of this phase would correspond to:
   “Tissue fragments showing notorious signs of maturation of vascular connective proliferation, with regression of inflammatory infiltration and capillary weft. Reinforcement of collagen fibers at the interstitial level.

Regarding the microbiological cultures carried out, the report of Staphylococcus aureus was obtained as the most frequent microorganism, followed by Staphylococcus epidermidis, Streptococcus spp, and coliform bacteria, which is consistent with reported findings for this kind of lesions and concomitant pathology, which is of interest, since polymicrobial infections can affect physiopathological aspects, which generates that this group of microorganisms complicates the injury recovery and healing. Indeed, cultures taken once the application of the gel has started were persistently negative.

The findings obtained in the current research could be explained by the cicatricial and bactericidal properties of the therapeutic product, which has the capacity to cause hyperplasia and increased keratinization. In the particular case of fibroblasts and endothelial cells, an increase in the production of cell surface proteins or fibronectin is observed, which provides the substrate substance required for the growth and differentiation of the epidermis. The results of this study prove the feasibility of using the product.

The revision of current literature does not show evidence of the use of the therapeutic proposal applied in this series of cases; that is the reason why it is considered a novel contribution in the attention of skin ulcer, which represents a serious problem of national and world public health. However, the results obtained coincide with the ones reported in similar studies.

### REFERENCES

1. Beltrán C, Fernández A, Giglio S et al. Tratamiento de la infección en el pie diabetico. Rev. Chil. Infectol. 18 (3): 212-224 (2001).
2. National Committee for Clinical Laboratory Standards. 2005.
3. Ramsey ds, Newton K. Blough. et al. Incidence, outcomes and cost of foot ulcers in patients with diabetes. Diab Care. 1999; 22: 382-387).
4. R. Olsen, D. Schwatzmüller, W. Weppner, “Biomedical Applications of Chitin and its Derivatives”, Physical Properties and Applications, pp. 813-828 (1989).
5. M. Nakajima, K. Atsumi, et al., “Development of Absorbable Sutures from Chitin”, pp. 407-11, Proceedings on Advances in Chitin, Chitosan, (1984).
6. Zikakis, J. P., (Ed.), “Chitin, Chitosan and Related Enzymes”, Academic Press, (1984).
7. J. Kost (Ed.), Pulsed and Self-Regulated Drug Delivery, CRC Press, Boca Raton, (1990).
8. K. E. Uhrich, S. M. Camizzaro, et al., Polymeric systems for controlled drug release, Chem. Rev., 99, 3181 (1999).
9. Wang X. & Spencer G., http://hubcap.clemson.edu/˜xwang/chitosan.htm, Department of Chemistry, Clemson University. USA.
10. Tomaszewska M., Chitin World, (Proc. Int. Conf. Chitin Chitosan), 6th, 583-589 (1994).
11. Hirano S. y Usutani A., Int. J. of Biological Macromolecules, 20, 245-249 (1997).
12. Rathke T. y Hudson S., J. M. S. Rev. Macromol. Chem. Phys., C34(3), 392-393 (1994).
13. Tokura S. et al, Macromol. Symp. 101, 389-396 (1996). 14. Pospieszny H., Crop Protection, 16, N^{o} 2, 105-106 (1997).
15. Staroniewicz Z. et al, Chitin World, (Proc. Int. Conf. Chitin Chitosan), 6th, 374-377 (1994).
16. Andrews, J. Determination of minimum inhibitory concentration. J. Antimicrob. Chemother, 48 (suppl 1): 5-16 (2001).
17. L. Y. Chung, R. J. Schmidt, et al., Journal of Biomedical Materials Research, Vol. 28, pp. 463-469

(1994).
18. S. M. Hudson, “Review of Chitin and Chitosan as Fiber and Film Former”, Journal of Material Science, C34(3), 375-437 (1994).
19. Concejo, J. Gonzalez, S. and Garcia, L. (2008) “Nanomedicina; el avance de la nanotecnología en la medicina”. Vol. 1, pp 47-56.
20. Org. Mundial de la Salud, Centro de prensa, nota descriptive N^{o} 297, “Cancer”, 2013 http://www.who.int/mediacentre/facts-heets/fs297/en/index/html (Consulta 9 de Julio de 2013).
21. a) Medina, C.; Santos-Martinez, M; Radomski, M. W. Chem. Rev. 2005, 125, 1547-1562.

b) Yih, T. C.; Wei, C. (2010) Nanomedicine in cancer treatment. Nanotechnol. Biol.Med. 1, 191-192
22. Bourne, (http://www.bourneresearch.com)
23. Chang, D, Yang, J y Zhao, Y. Chin. Med. Equip. 4: 26-32 (2004).
24. Cohen, M S, Stern, J M, Vanni, A J, Kelley, R S, Baumgart, R. Field, D, Libertino, J A y Summerhayes, J C Surg. Infect. 8: 397-402 (2007).
25. Panyman, J y Labhasetwar, V. Adv. Drug Delivery Rev. 55:3329-3347 (2003).
26. Elechiguerra, J. L., Burt, J., Morones, J R, Camacho-Bragado, A., Gao, X., Lara, H V. Y Yacaman, J. Nanobiotechnol. 3: 6 (2005).
27. Zhang, Y., Sun, J. Chin. J. Med. Instrum. 31: 35-38 (2007).
28. Lee, H., Park, H., Lee, Y M, Kim, K. y Park, S B. Chem. Commun. 28:2959-2961 (2007).
29. Iglesias Gonzalez R. Resumen de las recomendaciones de la American Diabetes Association (ADA) 2014 para la práctica clínica en el manejo de la diabetes mellitus. Diabetes Care. 2014;37: S14-80.
30. Serrano Hernando F J, Martín Conejero A. Rev Esp Cardiol. 2007; 60(9):969-82.
31. Vázquez-Hernandez I, Acevedo-Peña M. Prevalencia de insuficiencia venosa periférica en el personal de enfermería. Enfermería Universitaria. 2016; 13(3):166-170
32. Suthar, Manish, et al. Treatment of chronic non-healing ulcers using autologous platelet rich plasma: a case series. Journal of biomedical science. 2017; 24(1):16.
33. Nelson, T. G., et al. Cutaneous ulcers in a returning traveller: a rare case of imported diphtheria in the UK. Clinical and experimental dermatology. 2016; 41(1):57-59.
34. Lewis, Hal A., et al. “Methotrexate-induced cutaneous ulceration in 3 nonpsoriatic patients: Report of a rare side effect.” JAAD case reports. 2017; 3(3): 236-239.

### CONCLUSIONS

Chronic skin ulcers are a relevant health problem worldwide, it is estimated that 1% to 2% of the population will suffer the consequences thereof during their lifetime. In Chile, the available statistics show that approximately 160,000 patients suffer from some type of wound or ulcer, which represents a public health problem. Chronic ulcer is understood as “a skin injury maintained over time, involving loss of the epidermis, part of the dermis and even hypodermis”; accordingly, this group includes venous ulcers, ischemic ulcers, diabetic foot, pressure ulcers, burns and infected surgical wounds.

Chronic skin ulcers provide favorable conditions for microbial multiplication due to the loss of skin integrity, tissue hypoxia and contamination of exposed tissues, which hinder the healing thereof further to the increase in antibiotic resistance observed in frequently detected strains at least for usual antibiotic carriers. The above-mentioned arguments motivate the interest in knowing active principles of innovative use, which facilitate both the application, and stable and persistent effect on injured tissues, overlapping mechanisms of bacterial resistance, eventually enriched by antibiotics of known use.

Thus, the gel variants provided for this study have allowed observing an objectively favorable clinical progression, whose speed of recovery is greater than the one obtained with other therapeutic resources until the moment of receiving the application of the gel in an environment of high asepsis.

Accordingly, the character of the gel variants applied as a therapeutic agent showed that,
it is an agent that allows providing high levels of asepsis to the tissue injury along with histopathological neoangiogenesis, regressive changes in the cellular inflammatory infiltrate with appearance of fibroblastic response of a cicatricial nature and signs of complementary re-epithelialization.
it is an agent that tends towards safe, well-tolerated, satisfactory healing mechanisms providing benefits superior to the ones achieved with other therapeutic resources used in frequent polyclinic care.
it has features of ambulatory and autonomous application by the patients. As long as it can be complemented with eventual profile of massive access and accessible cost, it would be included in a potential group of profitable therapeutic resources and of high demand, which should be studied in the near future.

TABLE 1
Distribution of participants according to age and sex
(n = 30)

| Sex | |
|---|---|
| Male | Female |

| Age groups (years old) | No. | % | No. | % |
|---|---|---|---|---|
| 18-37 years old | 1 | 3.3 | | |
| 38-57 years old | 1 | 3.3 | 2 | 6.6 |
| ≥58 | 8 | 26.6 | 18 | 60.0 |
| Total | 10 | 33.3 | 20 | 66.6 |

TABLE 2
Frequency of morbid history in patients

(n = 30)

| Morbid history | No. | % |
|---|---|---|
| Arterial hypertension | 11 | 36.6 |
| Diabetes Mellitus | 12 | 40.0 |

Etiology

| | | |
|---|---|---|
| Peripheral arterial disease | 22 | 73.3 |
| Peripheral venous disease | 3 | 10.0 |
| Peripheral arterial disease | 4 | 13.3 |
| Peripheral venous disease | | |
| (mixed) | | |

TABLE 3
Frequency in skin-ulcer patients according to the score obtained
in the classification algorithm before the therapeutic protocol

| | |
|---|---|
| Classification of | (n = 30) |

| skin ulcer | No. | % |
|---|---|---|
| Type 1 | 1 | 3.3 |
| Type 2 | 3 | 10.0 |
| Type 3 | 5 | 16.6 |
| Type 4 | 21 | 70.0 |

TABLE 5
Analysis of skin ulcer progression before and after treatment

| | Therapeutic protocol | | | | | |
|---|---|---|---|---|---|---|
| | Before | | After | | | |
| Classification | N = 30 | | n = 30 | | Value | Value |
| of skin ulcer | No. | % | No. | % | p* | Z^{¥} |
| Type 1 | 1 | 3.3 | 5 | 16.6 | 0.00 | −6.042 |
| Type 2 | 3 | 10.0 | 6 | 20.0 | 0.00 | −3.842 |
| Type 3 | 5 | 16.6 | 7 | 23.3 | 0.01 | −3.624 |
| Type 4 | 21 | 70.0 | 12 | 40.0 | 0.01 | −1.342 |

*Probability value, Wilcoxon signed-rank test. 95% confidence interval (p ≤ 0.05).
^{¥}Value of the distribution of the standard, random and normal variable, Wilcoxon signed-rank test. 95% confidence interval (p ≤ 0.05).

TABLE 6
Progression of renal, hepatic and hematological
function in the studied patients

(n = 30)
Mean/Standard Deviation

| Analytical parameters | Day 0 | Day 42 |
|---|---|---|
| Leukocytes (cell/mm³) | 11.678 ± 2 | 8.677 ± 2 |
| Urea (mg/dL) | 163 ± 11 | 121 ± 10 |
| Creatinine (mg/dL) | 2.7 ± 0.5 | 2.0 ± 0.5 |
| GOT (U/I) | 62 ± 0.5 | 42 ± 5 |
| GPT (U/I) | 51 ± 2 | 43 ± 2 |
| GGT (U/I) | 49 ± 3.5 | 51 ± 3.5 |

Histopathological Progression

Material and Method:
1. Biopsy was obtained through punch modality, 2 mm in diameter, and 2-3 mm in depth.
2. Three biopsies were taken from each patient on days 1, 21, and 42. Each punch was immersed in 10% buffered formalin (25 cc).
3. Each sample was processed within 24 hours of the collection thereof.
4. Hematoxylin-eosin, Van Giesson and Giemsa stains were done for each inclusion.

Findings:
2. From the initial sample:
   a. In all cases, evaluable tissue material was observed.
   b. In 41.3% of the cases,
      i. superficial fibrinoleukocyte material (devitalized, purulent-type) was observed in more than 50% of the material evaluated.
      ii. In 24.1% of the cases, it corresponded to more than 75% of the material evaluated
   c. In 100% of the cases abundant inflammatory infiltration was observed, predominantly polymorphonuclear.
   d. In 27.5% of the cases, serum-leukocytary, and superficial hematic material (scabby) was observed.
   e. 10.3% of the cases showed signs of subepithelial sclerosis.
   f. 82.7% of the cases showed signs of deficient vascularization, for the clinical progression over time.
   g. 93.1% had intense clinical signs of an exudative nature, at least in a focal manner.
   h. 17.2% showed paratropic deposit of some substance, hemosiderine mainly.
   i. 48.2% of the cases examined showed microgranular hyperbasophilic accumulations of the bacterial colony type. In 6.8% of the cases, suggestive images of spores and hyphae were observed.
   j. The overall description of this phase would correspond to: “Tissue fragments showing abundant changes of exudative character, with polymorphonuclear predominance and frequent pyocytes that extensively infiltrate the samples, replacing their traditional architecture with accentuated signs of tissue lysis, foci of recent hemorrhage and signs of ancient hemorrhage. Rare vascular formations of capillary character and preserved structure are observed. Devitalized material of fibrinoleucocyte and serohematic character is observed on surface cover”.
   k. The overall diagnostic conclusion for this phase was: “Solution of tegumentary continuity with intense associated, active and chronic inflammatory process, whose diagnostic spectrum includes: diabetic foot ulcer, pustular folliculitis and fistulous tract, among others.
3. From the intermediate sample:
   a. In all cases, evaluable tissue material was observed.
   b. In 86.2% of the cases signs of variable intensity of fibroblastic proliferation of reparative character were observed.
   c. In 27.5% of the cases signs of peripheral, immature and incipient re-epithelization were observed (edges of the ulcer).
   d. In 65.5% of the cases,
      i. inflammatory infiltration of predominantly mononuclear, lymphoplasmacytic of chronic type was observed in more than 50% of the evaluated material.
      ii. In 41.3% of the cases, it corresponded to more than 75% of the material evaluated
   e. 27.5% of the cases showed signs of subepithelial sclerosis.
   f. In 86.2% of the cases recovery of vascular pattern, and signs of capillary and endothelial proliferation were observed, which showed signs of mild to moderate edema (neoangiogenesis).
   g. 93.1% of the cases did not show signs of superficial fibrinoleucocyte material, or serohematic material (superficial and scabby).
   h. No hyperbasophilic microgranular accumulations, such as bacterial colonies or images suggesting spores and hyphae were observed in any of the cases examined.
   i. The overall description of this phase would correspond to: “Tissue fragments showing signs of connective-vascular proliferation of reparative character, characterized by the installation of congestive capillary network, chronic inflammatory infiltration of predominantly lymphoplasmacytic complemented with less polymorphonuclear component. No devitalized material or signs of microorganisms are observed.
   j. The overall diagnostic conclusion for this phase was: “Solution of tegumentary continuity, with development of granulated, congested tissue. There are no signs of infection. Incipient peripheral signs of re-epithelization, involving incomplete cell differentiation”.
4. From the final sample:
   a. In all cases, evaluable tissue material was observed.
   b. 96.5% of the cases showed signs of regression of inflammatory infiltration located at the perivascular level mostly.
   c. In 93.1% of cases, no superficial fibrinoleucocyte material (devitalized purulent-type) or superficial hematic material (scabby type) was observed.
   d. 100% of the cases showed signs of reduplication of collagen fibers in interstitial matrix.
   e. 100% of the cases showed signs of capillary weft.
   f. No hyperbasophilic microgranular accumulations, such as bacterial colonies or images suggesting spores and hyphae were observed in any of the cases examined.
   g. The overall description of this phase would correspond to: “Tissue fragments showing notorious signs of maturation of vascular connective proliferation, with regression of inflammatory infiltration and capillary weft. Reinforcement of collagen fibers at the interstitial level.
   h. The overall diagnostic conclusion for this phase was: “Solution of tegumentary continuity exhibiting scar tissue changes. Signs of re-epithelialization showing adequate tissue differentiation. No microorganisms are observed”.

1. Method for preparing a biodegradable and biocompatible gel, CHARACTERIZED in that said method includes the following steps:
i) preparation of chitosan gels:
a chitosan polymer with a deacetylation degree higher than 90% and a molar mass between 25,000 and 500,000 g/mol is used, which is obtained by dissolution at room temperature (15-25° C.) in weak organic acids (acetic acid, formic acid, lactic acid, among others) until a concentration between 1 and 5% by mass is achieved;
ii) preparation of chitosan mixed with antibiotics:
one or more antibiotics are added to the chitosan matrix obtained in (i) by the method of dispersion and agitation in a gaseous nitrogen atmosphere, wherein the antibiotics are at a concentration of 0.1 to 5% by mass and in a ratio of 1:1, if combined;
iii) preparation of nanoparticles:
co-deposition reaction: this method involves the physical co-deposition of metal vapors (copper and silver) with organic vapors (2-ethoxyethanol) in liquid nitrogen, in a reactor of metal atoms; the metal to be evaporated is introduced into an alumina crucible;
the magnet and the solid chitosan are introduced into the bottom of the reactor; the flask is connected to the solvent to be used (2-ethoxyethanol) and the whole system is evacuated until a high vacuum of 10⁻⁵ bar is reached. The reactor is then immersed in a 5000 ml Dewar with liquid nitrogen. The power source is turned on up to 40A, which evaporates both the solvent and then the metal at its respective boiling temperature, penetrating the solvent into the reactor in vaporous state. The power source is turned off, the metal and the solvent are co-deposited in a process called nucleation for a period of approximately 60 minutes, finally forming a frozen “metal-organic” matrix on the internal walls of the reactor;
iv) preparation of chitosan doped with nanoparticles The chitosan doped with the metallic nanoparticles is obtained by defrosting the “metal-organic” matrix found on the internal walls of the reactor. The matrix is shaken therein for 12 hours with a magnetic stirrer, and it rests for a period of approximately 2 hours, thus obtaining the “metal-organic-polymer” matrix from which it is extracted by connecting a receiving flask to the vacuum line. Once the matrix is removed, the solvent is evaporated, which acts as a support for the nanoparticle/chitosan mixture until solid chitosan particles are obtained. These particles are doped with metallic nanoparticles, wherein said particles are in a concentration of 1 to 5% by mass with respect to the chitosan polymer.

2. The method for preparing a biodegradable and biocompatible gel, according to claim 1, CHARACTERIZED in that the antibiotics correspond to ciprofloxacin and/or cloxacillin alone or in a mixture thereof and/or cefotaxime and/or gentamicin alone or in a mixture thereof.

3. The method for preparing a biodegradable and biocompatible gel, according to claim 1, CHARACTERIZED in that the nanoparticles correspond to copper and/or silver alone or in a mixture thereof.

4. A biodegradable and biocompatible gel, CHARACTERIZED in that said gel comprises a chitosan polymeric matrix in a concentration of 1 to 5% by mass, containing metallic nanoparticles in a range from 10 to 100 nm, in a concentration of 1 to 5% by mass and/or containing antibiotics in a concentration of 0.1 to 5% by mass and additives between 1 to 5% by mass.

5. Gel according to claim 4, CHARACTERIZED in that the chitosan corresponds to poly-beta-D-glucosamine and the molar mass of the polymeric matrix ranges between 25,000 and 500,000 g/mol with a degree of purity greater than 95% by mass.

6. Gel according to claim 4, CHARACTERIZED in that the metallic ions correspond to copper and/or silver alone or in a mixture thereof.

7. Gel according to claim 4, CHARACTERIZED in that the antibiotics correspond to ciprofloxacin and/or cloxacillin alone or in a mixture thereof and/or cefotaxime and/or gentamicin alone or in a mixture thereof.

8. Gel according to claim 4, CHARACTERIZED in that the combined antibiotics are in a 1:1 ratio.

9. Gel according to claim 4, CHARACTERIZED in that the additives correspond to carbopol, hydroquinone, guar gum, xanthan gum and/or gum arabic.

10. Gel according to claim 4, CHARACTERIZED in that it is useful for treating bacterial skin infections whether they are neuropathic ulcers or diabetic foot.

11. Gel according to claim 4, CHARACTERIZED in that it is useful for the restoration of epidermal and dermal tissues, neovascularization and angiogenesis.

12. Gel according to claim 4, CHARACTERIZED in that it is useful as a matrix for fibroblast cell growth in epidermal and dermal tissues.
